(19) United States
(12) Patent Application Publication
(10) Pub. No.: US 2007/0275064 A1
(43) Pub. Date: Nov. 29, 2007

(54) Solid Tablets Composition Rapidly Dissolvable in Water or Watery Liquids and Used for Body, Skin, Hair and Oral Hygiene and Desinfection

(76) Inventor: Jose Alejandro MUMOLI, Buenos Aires (AR)

Correspondence Address: FITCH EVEN TABIN AND FLANNERY, 120 SOUTH LA SALLE STREET, SUITE 1600, CHICAGO, IL, 60603-3406, US

(21) Appl. No.: 11/753,879

(22) Filed: May 25, 2007

(30) Foreign Application Priority Data
May 29, 2006 (AR) 060102213

(51) Int. Cl. A61K 36/886; A61K 31/155; A61K 9/22; A61K 36/889; A61K 36/82; A61K 36/736; A61K 36/752; A61K 36/61; A61K 31/14

(52) U.S. Cl. 424/468; 424/78.3; 514/642; 514/634; 424/195.17; 424/745; 424/746; 424/757; 424/764; 424/735; 424/736; 424/765; 424/727; 424/742; 424/744; 424/778; 424/729

(57) ABSTRACT

Compositions of solid tablets of quick dissolution in water or aqueous liquids for body or mouth cleansing, skin and hair treatment, and disinfection, wherein said tablet composition comprises from 75% to 92% of at least one disintegrating agent, from 0.5% to 9% of at least one surfactant and from 0.5% to 4% of at least one disgregating agent.

This invention refers to compositions of solid tablets of quick dissolution in water or aqueous liquids for body or mouth cleansing, skin and hair treatment, and disinfection, wherein said tablet composition comprises from 75% to 92% of at least one disintegrating agent, from 0.5% to 9% of at least one surfactant and from 0.5% to 4% of at least one disgregating agent.

### BACKGROUND OF THE INVENTION

Most of the body or mouth cleansing agents, i.e. soaps, tooth pastes; or for hair cleansing, such as capilar baths or for pediculosis treatment, are creams, liquids, gels or pastes which contain different proportions of water.

U.S. Pat. No. 6,541,441 to Mumoli discloses quick dissolution individual soaps comprising sodium lauryl sulfate, binding agents and two different disintegration agents, wherein sodium lauryl sulfate is present in a quantity comprised from 15 wt % to 35 wt %, a first disintegrating agent consisting of crosscarmellose in a quantity from 15 wt % to 25 wt %, and a second disintegrating agent consisting of microcrystalline cellulose in a quantity from 50 wt % to 60 wt %.

U.S. Pat. No. 6,664,225 to Mumoli discloses a single-dose cleansing and treating agent which comprises from 10 wt % to 30 wt % of a surfactant, from 5 wt % to 20 wt % of a binding agent, from 5 wt % to 15 wt % of a first disintegrating agent and from 10 wt % to 60 wt % of a second disintegrating agent, from 3 wt % to 20% of a phyto-therapeutical extract and bactericides, dyes and essences.

### BRIEF DESCRIPTION OF THE INVENTION

Proportion of basic components of the solid tablet composition of this invention is such that it enhances hardness and reduces friability thereof as compared to the previous art, showing advantages regarding a lower loss of material during its handling and packing, whilst retaining its properties in terms of hygiene and quick dissolution. All percentages are expressed as weight percentage on the basis of the composition total weight.

It is an object of this invention to provide a composition of solid tablets of quick dissolution in water for body and mouth cleansing, skin treatment and disinfection, which comprises from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate; cetrimide or combinations thereof; and the disgregating agent is for example crosscarmellose, carboximethylcellulose, polyvinyl-pyrrolidone, starch, Carbopol, carrageenan, or combinations thereof. The composition may also comprise other products, for instance vegetal oils, antibacterial agents, antiimflammatory agents, plant extracts, insecticides, pH buffers, sweeteners, flavours, fluorides, vitamins, dyes, insect-repelling agents, perfumes, antiperspirant agents, antifungal agents, decongestive agents, abrasives, disinfectants, effervescing agents, tartar inhibitors, or combinations thereof.

It is still another object of the present invention to provide a composition of solid tablets of quick dissolution in water for body hygiene comprising from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, Cetrimide or combinations thereof; and the disgregating agent is for example crosscarmellose, carboximethylcellulose, polyvinyl-pyrrolidone, starch, Carbopol or combinations thereof. The composition may also comprise other products, for instance vegetal oils, antibacterial agents, antiimflammatory agents, plant extracts, dyes, perfumes, anti-perspirant agents, antifungal agents, decongestive agents, disinfectants, or combinations thereof.

It is still another object of the present invention to provide a composition of solid tablets of quick dissolution in water for mouth hygiene comprising from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate or combinations thereof; and the disgregating agent is for example crosscarmellose, carboximethylcellulose, polyvinylpyrrolidone, starch, Carbopol or combinations thereof. The composition may also comprise other products, for instance vegetal oils, antibacterial agents, plant extracts, sweeteners, flavours, fluorides, vitamins, dyes, abrasives, disinfectants, effervescing agents, tartar inhibitors, or combinations thereof.

It is still another object of the present invention to provide a composition of solid tablets of quick dissolution in water for skin treatment comprising from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, Cetrimide or combinations thereof; and the disgregating agent is for example crosscarmellose, carboximethylcellulose, polyvinyl-pyrrolidone, starch, Carbopol, carrageenan or combinations thereof. The composition may also comprise other products, for instance vegetal oils, antibacterial agents, antiimflammatory agents, plant extracts, antifungal agents, decongestive agents or combinations thereof.

It is still another object of the present invention to provide a composition of solid tablets of quick dissolution in water for repelling insects comprising from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, Cetrimide or combinations thereof; and the disgregating agent is for example crosscarmellose, carboximethylcellulose, polyvinyl-pyrrolidone, starch, Carbopol, carrageenan or combinations thereof. The composition may also comprise other products, for instance vegetal oils, plant extracts, dyes, insect-repelling agents, perfumes or combinations thereof.

It is still another object of the present invention to provide a composition of solid tablets of quick dissolution in water for disinfection and pediculosis treatment comprising from 75% to 92% of at least one disintegrating agent, from 0.5% to 9% of at least one surfactant and from 0.5% to 4% of a least one disgregating agent. Said disintegrating agent may be, for example, microcrystalline cellulose, amylose, cellactose, powder cellulose, sugars, or combinations thereof; surfactant is, for example, sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, Cetrimide or combinations thereof; and the disgregating agent is for example crosscarmellose, carboximethylcellulose, polyvinylpyrrolidone, starch, Carbopol or combinations thereof. The composition may also comprise other products, for instance vegetal oils, plant extracts, insecticides, pH buffers, dyes, perfumes or combinations thereof.

### DETAILED DESCRIPTION OF THE INVENTION

Within the context of the present invention, “disintegrating agent” refers to those substances into which water enters and forms channels; for example, disintegrating agents include, without limitation, microcrystalline cellulose, amylose, cellactose, powder cellulose, and sugars.

Within the context of the present invention, “disgregating agent” refers to a substance which upon its contact with water increases its volume causing bursting of the whole. Disgregating agents include, without limitation, sodium crosscarmellose, carboximethylcellulose (CMC), polyvinylpyrrolidone (PVP), starch, gelatines and Carbopol.

Within the context of the present invention a “surfactant” is a surface active agent which reduces surface tension. For instance, surfactants may be, without limitation, sodium lauryl sulfate and sodium cocoyl isethionate.

The inventive tablet composition is meant for external use only. Said tablets are powder tablets and thence they do not contain water or other aqueous solutions. Due to the lack of water in the composition, tablets may remain packed during long periods of time without requiring preservatives to be added.

The inventive tablet composition may be individually packed.

The inventive composition of solid tablets of quick dissolution in water for body and mouth hygiene, skin treatment and disinfection comprises from 75% to 92% of at least one disintegrating agent, from 0.5% to 9% of at least one surfactant and from 0.5% to 4% of a least one disgregating agent. This composition exhibits excellent hardness parameters with friability indexes lower than 1% which facilitate handling thereof and reduce material wastes. Different compounds may be added to the composition according to the invention, such as perfumes, dyes, bactericide agents, antifungal agents and so on.

In a preferred embodiment, the tablet for hands and body cleansing may comprise at least a mixture of at least one surfactant, at least a disintegrating agent and at least a disgregating agent. As an example, there follow different tablets compositions for hands and body cleansing.

Tablet Composition No. 1

| | |
|---|---|
| Sodium lauryl sulfate | 0-8% |
| Sodium cocoyl isethionate | 0-8% |
| Microcrystalline cellulose | 80-92% |
| Sodium crosscarmellose | 1-4% |

Tablet Composition No. 2

| | |
|---|---|
| Sodium cocoyl isethionate | 8-9% |
| Microcrystalline cellulose | 82-90% |
| Sodium crosscarmellose | 0.5-3% |
| Carboximethylcellulose (CMC) | 0.5-3% |
| Vegetal oils | 0.5-1.5% |
| Dyes | 0.1-0.5% |

Tablet Composition No. 3

| | |
|---|---|
| Sodium lauryl sulfate | 4-8% |
| Triclosan | 0.1-0.3 |
| Chlorhexidine | 0.1-0.5% |
| Sodium crosscarmellose | 1-4% |
| Microcrystalline cellulose | 87-92% |

Tablet Composition No. 4

| | |
|---|---|
| Sodium lauryl sulfate | 0-8% |
| Sodium cocoyl isethionate | 0-8% |
| Triclosan | 0.1-0.3% |
| Iodine Polyvinylpyrrolidone | 2-8% |
| Microcrystalline cellulose | 80-90% |
| Sodium crosscarmellose | 1-4% |

This composition is particularly useful for hands and body asepsis.

Tablet Composition No. 5

| | |
|---|---|
| Sodium lauryl sulfate | 2-6% |
| Aluminum hydrochloride | 7-14% |
| Cyclometicone | 3-5% |
| Salvia officinalis extract | 1-3% |
| Microcrystalline cellulose | 75-86% |
| Sodium crosscarmellose | 1-4% |
| Perfume | 0.5-2% |
| Dye | 0.1-0.5% |

This composition is particularly useful for its application onto the body as antiperspirant soap.

Tablet Composition No. 6

| | |
|---|---|
| Sodium lauryl sulfate | 3-8% |
| Salvia extract | 1-3% |
| Myconazol nitrate | 1-2% |
| Microcrystalline cellulose | 82-92% |
| Carboximethylcellulose | 1-4% |

This composition is particularly useful for its use as a tablet for corporal cleansing with antifungal properties.

Tablet Composition No. 7

| | |
|---|---|
| Sodium cocoyl isethionate | 8-9% |
| Licorice extract | 0.5-2% |
| Alantoine | 0.5-3% |
| Green tea extract | 0.5-3% |
| Perfume | 1-2% |
| Dye | 0.2-0.5% |
| Microcrystalline cellulose | 75-88% |
| Sodium crosscarmellose | 3-4% |

This composition is useful for the manufacturing of face cleansing tablets and shaving cream.

Tablet Composition No. 8

| | |
|---|---|
| Sodium cocoyl isethionate | 3-8% |
| Polyvinylpyrrolidone | 1-4% |
| Vegetable fucus oil | 0.5-2% |
| Aloe Vera oil | 0.5-3% |
| Microcrystalline lactose and cellulose | 75-92% |
| Perfume | 0.5-2% |
| Dye | 0.02-0.5 |

Due to its cleansing and protecting properties, this composition is particularly useful as hair bath.

Tablet Composition No. 9

| | |
|---|---|
| Sodium cocoyl isethionate | 1-3% |
| Hydrolysed collagen | 1-3% |
| Alantoine | 0.5-3% |
| Polyvinylpyrrolidone | 1-3% |
| Microcrystalline lactose and cellulose | 75-92% |
| Perfume | 0.5-1.5% |
| Dye | 0.01-0.5 |

This composition is particularly useful for face cleansing and further, as antiaging cream.

Tablet Composition No. 10

| | |
|---|---|
| Sodium lauryl sulfate | 3-9% |
| Carboximethylcellulose | 0.5-3% |
| Acetylsalicylic acid | 0.5-3% |
| Microcrystalline lactose and cellulose | 75-90% |
| Starch | 0.5-2% |
| Dye | 0.001-0.05 |
| Perfume | 0.2-1% |

This composition is particularly useful as hair bath due to its cleansing, protection and anti-inflammatory properties.

In another preferred embodiment, the inventive tablet may comprise at least a mixture of at least a surfactant, at least a disintegrating agent and at least a disgregating agent. The following are examples of different tablet compositions for mouth cleansing

Tablet Composition No. 11

| | |
|---|---|
| Chlorhexidine | 0.05-0.2% |
| Triclosan | 0.05-0.2% |
| Sodium lauryl sulfate | 1-4% |
| Zinc citrate | 0.03-0.08% |
| Benzosulfamide | 0.5-3% |
| Mint | 1-5% |
| Lactose | 85-90% |
| Sodium crosscarmellose | 1-3% |

Tablet Composition No. 12

| | |
|---|---|
| Sodium lauryl sulfate | 1-4% |
| Triclosan | 0.05-0.3% |
| Nicomethanol fluorhydrate | 0.05-0.5 |
| Panthenol (Provitamin B5) | 0.2-1% |
| Microcrystalline cellulose | 85-92% |
| Sodium crosscarmellose | 1-4% |
| Dye | 0.1-0.5 |
| Menthol | 1-3% |
| Sodium Saccharine | 0.8-2% |

Tablet Composition No. 13

| | |
|---|---|
| Sodium lauryl sulfate | 1-3% |
| Chlorhexidine | 0.005-0.2% |
| Xylitol | 0.5-1.5% |
| Micronized sodium bicarbonate | 0.5-8% |
| Carboximethylcellulose | 0.5-3 |
| Amylose and microcrystalline cellulose | 75-92% |
| Flavour - Cherry oil | 0.5-3% |
| Dye | 0.1-0.5% |

Compositions 11, 12 and 13 are particularly useful as toothpastes. Composition is applied on the toothbrush and this is used as any other commercial paste dentifrice.

Tablet Composition No. 14

| | |
|---|---|
| Sodium N-laurylsarcosinate | 0.2-3% |
| Sodium monofluor fosfate | 0.05-0.3% |
| Triclosan | 0.05-0.2% |
| Microcrystalline cellulose | 88-92% |
| Sodium crosscarmellose | 1-4% |
| Eucalyptus oil | 0.05-2% |
| Dye | 0.05-02% |
| Sodium Saccharine | 0.5-2% |

Tablet Composition No. 15

| | |
|---|---|
| Sodium lauryl sulfate | 0.5-3% |
| Hexetidine | 0.2-0.5% |
| Zinc lactate | 0.1-0.5% |
| Sodium saccharine | 0.2-2% |
| Microcrystalline lactose and cellulose | 75-92% |
| Sodium bicarbonate | 5-10% |
| Sodium crosscarmellose | 0.5-4% |
| Citrus extract | 1-3% |
| Menthol | 0.2-0.5% |
| Dye | 0.01-0.05% |

Compositions 14 and 15 are used as mouthwash, for example, the inventive tablet composition is introduced into a water-containing glass and then it is applied as a mouthwash.

In another preferred embodiment of the tablet of the invention, it may comprise a mixture of at least one surfactant, at least one disintegrating agent and at least a disgregating agent. There follow examples of different tablet compositions that may be manufactured for skin treatment purposes, as in the case of acne, dermatitis or dermatosis.

Tablet Composition No. 16

| | |
|---|---|
| Sodium cocoyl isethionate | 2-8% |
| PVP - Iodine | 2-6% |
| Alantoine | 1-4% |
| Salicylic acid | 0.5-3% |
| Betula Alba - birch | 1-5% |
| Microcrystalline cellulose | 75-90% |
| Sodium crosscarmellose | 1-4% |

Tablet Composition No. 17

| | |
|---|---|
| Sodium cocoyl isethionate | 0.5-6% |
| Betamethasone dipropionate | 0.2-0.8% |
| Clotrimazole | 0.8-1.2% |
| Gentamicine | 0.1-0.15% |
| PVP | 1-4% |
| Microcrystalline cellulose | 89-92% |

Tablet Composition No. 18

| | |
|---|---|
| Sodium cocoyl isethionate | 0.5-9% |
| Chlorhexidine diglutamate | 0.2-0.5% |
| Salicylic acid | 0.5-3% |
| Mandelic acid | 0.5-6% |
| Aloe ferox Mill oil | 1-3% |
| Microcrystalline cellulose | 75-86% |
| Starch | 1-4% |

In another preferred embodiment the inventive tablet may comprise at least a mixture of at least one surfactant, at least one disintegrating agent and at least a disgregating agent. There follow examples of different tablet compositions that may be manufactured for corporal applications, which compositions exhibit insect-repellence properties.

Tablet Composition No. 19

| | |
|---|---|
| Sodium lauryl sulfate | 4-8% |
| DEET | 7-20% |
| Soybean oil | 0.5-3% |
| Perfume | 0.5-1.5% |
| Dye | 0.1-0.5% |
| Microcrystalline cellulose | 75-84% |
| Sodium crosscarmellose | 2-4% |

Tablet Composition No. 20

| | |
|---|---|
| Sodium lauryl sulfate | 0.5-3% |
| picaridine | 7-15% |
| P-menthanediol (PMD) | 1-3% |
| Eucalyptus-Lemon oil | 0.5-2% |
| Dye | 0.1-0.5% |
| Microcrystalline cellulose | 75-80% |
| Sodium crosscarmellose | 1-3% |

Tablet Composition No. 21

| | |
|---|---|
| Sodium cocoyl isethionate | 0.5-7% |
| N,N-diethyl-m-toluamide (DEET) | 8-15% |
| Aloe vera oil | 1-3% |
| Soybean oil | 1-3% |
| Microcrystalline cellulose | 75-80% |
| Sodium crosscarmellose | 0.5-4% |

Tablet compositions used as insect repellents are applied on the body as soap.

In another preferred embodiment the inventive tablet may comprise at least a mixture of at least one surfactant, at least one disintegrating agent and at least a disgregating agent. There follow examples of different tablet compositions that may be manufactured to be applied on the hair, which tablets have insecticide activity and are useful as, for example, pediculicides.

Tablet Composition No. 22

| | |
|---|---|
| Sodium cocoyl isethionate | 4-8% |
| Carboximethylcellulose | 1-4% |
| Lindane | 0.3-1.5% |
| Permethrin | 0.3-2% |
| Micronised citric acid | 3-8% |
| Microcrystalline cellulose | 75-85% |

Selected final presentation form of the product is the pharmaceutical form known as tablet for topic or external use.

In order to prepare the tablet composition of the invention, characteristics, compatibilities, quantities, particle size distribution and manufacturing forms have been studied for each component.

Typically, manufacturing process follows the following steps:

1. Definition of the exact proportion of each of the components;

2. Definition of the industrial batch to be prepared (for example 100 kg);

3. Weighting and fractionation of ingredients;

4. Premixing;

5. Mixing;

6. Sieving; and

7. Compression.

Preferably, the manufacturing process is that known as “direct compression”, wherein powder components are mixed and then compressed. Should wet compounds be incorporated the procedure is carried out by introducing stages such as wet absorption, wet granulation, drying and milling, or others as may correspond.

All of the elaboration and packing process is carried out in a controlled atmosphere environment, with temperatures of about 20/24° C. and RH of 40/55%.

Unless otherwise stated, fragrances o perfumes employed are in powder form, by wet absorption of essential oil on microcrystalline cellulose

Employed dyes are those known as water insoluble Aluminum Lakes, except mouthwashes which use water soluble pigments, in any case same are supplied in powder form.

This invention shall be better illustrated by means of the following examples, which are not to be considered as a limitation to the scope thereof. On the contrary, it should be clearly understood that other embodiments, changes and equivalents thereof that those skilled in the art may infer after reading the present description, without departing from the spirit of the present invention and/or the scope of the accompanying claims.

### EXAMPLES

### Example 1

### Tablet Composition No. 1 Elaboration

In order to prepare tablet composition No. 1, 4.5 kg of sodium lauryl sulfate, 4.5 kg of sodium cocoyl isethionate, 90.5 kg of microcrystalline cellulose and 0.5 kg of sodium crosscarmellose are weighted. The resulting mixture is placed in a mixer (of the double cone mixer or pony mixer or type “V” mixer, or the like) and it is mixed during 20′. Then, the mixture is sieved by means of a US 40 mesh oscillating type sieve and introduced into the bin of a multiple-punch rotary tableting machine in order to obtain 750 mg tablets of 13 mm diameter, hardness of about 4.5-6 Kp and a friability of about 1%.

### Example 2

### Tablet Composition No. 2 Elaboration

A premix is prepared by mixing in a small mixer 26.4 kg of microcrystalline cellulose, 1 kg of sodium crosscarmellose, 1 kg of CMC, 0.8 kg of perfume and 0.2 kg of dye, during 10′. Then, the premix is placed into a larger mixer and 9 kg of sodium cocoyl isethionate and 61.6 kg of microcrystalline cellulose are added, mixing during 20′. The resulting mixture is sieved by means of a US 40 mesh sieve and then compressed obtaining tablets with 13 mm diameter and 700/750 mg. Tablets exhibit a hardness of 4/7 Kp, friability of 1% or less and disgregation time shorter than 15 sec.

### Example 3

### Tablet Composition No. 3 Elaboration

A premix is prepared by mixing in a small mixer 27 kg of microcrystalline cellulose, 1.5 kg of sodium crosscarmellose, 0.2 kg of chlorhexidine and 0.3 kg of Triclosan during 10′. Then, the premix is placed into a larger mixer and 63 kg of microcrystalline cellulose and 8 kg of sodium lauryl sulfate are added. These are mixed during 20′. The resulting mixture is sieved by means of a US 40 mesh sieve and then compressed obtaining tablets with 13 mm diameter and 750 mg. Tablets exhibit a hardness of 4/7 Kp, friability of 1% or less and disgregation time shorter than 15 sec.

### Example 4

### Tablet Composition No. 4 Elaboration

A premix is prepared by mixing in a small mixer 25.26 kg of microcrystalline cellulose, 0.3 kg of Triclosan, 5 kg of PVP-1 and 1.5 kg of sodium crosscarmellose, during 12′. Then, the premix is transferred to a larger mixer and 58.94 kg of microcrystalline cellulose, 6 kg of sodium cocoyl isethionate and 3 kg of sodium lauryl sulfate are added, mixing during 20′. The resulting mixture is sieved by means of a US 40 mesh sieve and then compressed obtaining tablets with 13 mm diameter and 750 mg. Tablets exhibit a hardness of 5/7 Kp, friability of 1.2% or less and disgregation time shorter than 10 sec.

### Example 5

### Tablet Composition No. 5 Elaboration

A premix is prepared by mixing in a small mixer 22.47 kg of microcrystalline cellulose, 0.3 kg of powder cyclometicone, 0.1 kg of salvia officinalis extract previously absorbed in microcrystalline cellulose, 1.5 kg of sodium crosscarmellose, 1.5 kg of perfume and 0.1 kg of blue No. 4 aluminum lake, during 15′. Then, the premix is placed into a larger mixer and 52.43 kg of microcrystalline cellulose, 12 kg of aluminum chlorhydrate, and 6 kg of sodium lauryl sulfate are added, mixing during 30′. The resulting mixture is sieved by means of a US 40 mesh sieve and then compressed obtaining tablets with 16 mm diameter and 1 g. Tablets exhibit a hardness of 7 to 10 Kp, friability of 1.2% or less and disgregation time shorter than 15 sec.

### Example 6

### Tablet Composition No. 6 Elaboration

In order to prepare tablet composition No. 6, 3 kg of sodium lauryl sulfate, 3 kg of Salvia extract previously absorbed in microcrystalline cellulose, 2 kg of miconazole nitrate, 2 kg of carboximethylcellulose (CMC) and 90 kg of microcrystalline cellulose are weighted. All of the preparation is mixed in a double cone mixer or the like during 30′. Then, the resulting mixture is sieved by means of a US 40 mesh sieve and compressed with punches of 13 mm diameter and 72 mg, hardness being 4/7 Kp and friability of less than 1%, disgregation time less than 10 sec.

### Example 7

### Tablet Composition No. 7 Elaboration

A premix is prepared by mixing in a small mixer 24.54 kg of microcrystalline cellulose, 3 kg of sodium crosscarmellose, 0.2 kg de aluminum lake, 1 kg of oil perfume previously absorbed in microcrystalline cellulose, 1 kg of green tea extract previously absorbed in microcrystalline cellulose, 3 kg of powder alantoine, 2 kg of licorice extract previously absorbed in microcrystalline cellulose, during 10/15′. Then, the premix is placed into a larger mixer and 57.26 kg of microcrystalline cellulose and 8 kg of sodium cocoyl isethionate are added, mixing during 30′. The resulting mixture is sieved by means of a US 40 mesh sieve and then compressed by means of ⅝″ punches; weight 1 g, hardness 7-9 Kp, friability of less than 1.5% and disgregation time shorter than 20 sec.

### Example 8

### Tablet Composition No. 8 Elaboration

A 100 kg industrial batch is prepared.

A premix is prepared by mixing in a small mixer 15 kg of microcrystalline cellulose, 5 kg of lactose; 1.2 kg of perfume with violet essential oil previously absorbed in microcrystalline cellulose, 2 kg of mucus oil previously absorbed in microcrystalline cellulose, 2.5 kg of aloe vera oil previously absorbed in microcrystalline cellulose, and 0.02 kg of No. 2 yellow aluminum lake, during 20′. Then, the premix is placed into a larger mixer and 19.6 kg of lactose, 42.68 kg of microcrystalline cellulose, 4 kg of PVP-K30 and 8 kg of sodium cocoyl isethionate are added, mixing during 30′.

The resulting mixture is sieved by means of a US 40 mesh sieve. The resulting powder is compressed into Ø 22 mm tablets weighting 1.8 g each, hardness 7-10 Kp, friability of less than 1.5% and disgregation time shorter than 20 sec.

### Example 9

### Tablet Composition No. 9 Elaboration

A premix is prepared by mixing in an adequate mixer 1.5 kg of hydrolysed powder collagen, 2.5 kg of alantoine, 2 kg of PVP-K30, 17.99 kg of anhydrous lactose, 1 kg of powder rose perfume and 0.01 kg dye, during 20′. Then, the resulting mixture is placed into a larger mixer and 72 kg of microcrystalline cellulose and 3 kg of sodium cocoyl isethionate are added, mixing during 30′. All of the resulting mixture is sieved by means of a US 40 mesh sieve.

The resulting powder is compressed into Ø 13 mm tablets weighting 750 mg. They exhibit a hardness of 5-7 Kp, a friability of less than 1% and disgregation time shorter than 15 sec.

### Example 10

### Tablet Composition No. 10 Elaboration

A premix is prepared by mixing in an adequate mixer 15 kg of lactose, 2 kg of carboximethylcellulose, 0.8 kg acetylsalicylic acid, 1 kg of starch (Starch 1500), 0.01 kg of No. 4 blue aluminum lake, and 1 kg of perfume, during 15-20′. Then, the resulting mixture is placed into a larger mixer and 9 kg of sodium lauryl sulfate and 71.19 kg of microcrystalline cellulose. Mixing is performed during 30′ and subsequently the resulting powder is sieved through a US 30/40 mesh sieve.

The later is compressed into Ø 8 mm tablets weighting 900/950 mg, which exhibit a hardness of 5-8 Kp, a friability of less than 1.2% and disgregation time shorter than 15 sec.

### Example 11

### Tablet Composition No. 11 Elaboration

A premix is prepared by mixing in an small mixer 12.5 kg of anhydrous lactose, 0.1 kg of powder chlorhexidine, 0.2 kg of Triclosan, 0.05 kg of zinc citrate, 2 kg of benzosulfamide, 3 kg of mint flavour powder and 2 kg of sodium crosscarmellose, during 20′. Then, the resulting premix is placed into a larger mixer and 77.15 kg of anhydrous lactose and 3 kg of sodium lauryl sulfate are added. These are mixed during 20/30 minutes, the mixture is removed and sieved through a US 40 mesh. Compression is performed with oblong punch assembly (10×5). Weight: 400 mg, hardness: 3-5 Kp, friability: less than 1.5% and disgregation time shorter than 15 sec.

### Example 12

### Tablet Composition No. 12 Elaboration

A 100 kg mixture is prepared.

A premix is prepared by mixing in an small mixer 0.1 kg of Triclosan, 0.05 kg of nicomethanol fluorhydrate, 0.2 kg of panthenol, 5.05 kg of microcrystalline cellulose, 1 kg of sodium crosscarmellose, 0.1 kg of green aluminum lake, 2.5 kg of micronised menthol and 2 kg of sodium saccharine, during 15-20 minutes. The resulting premix is placed into a larger mixer and 86 kg of microcrystalline cellulose and 3 kg of sodium lauryl sulfate are added. These are mixed during 20/30 minutes, the resulting mixture is sieved through a US 30/40 mesh.

These are compressed into tablets of Ø 9 mm, weight 450 mg, hardness 4-7 Kp, friability less than 1.5% and disgregation time shorter than 15 sec.

### Example 13

### Tablet Composition No. 13 Elaboration

A premix is prepared by mixing in an small mixer 5 kg of amylose, 2 kg of sodium lauryl sulfate, 0.1 kg of chlorhexidine, 1.5 kg of xylitol, 3 kg of micronised sodium bicarbonate, 3 kg of carboximethylcellulose, 2 kg of cherry flavour oil previously absorbed in microcrystalline cellulose and 0.1 kg of aluminum red lake, during 15-20 minutes. The resulting premix is transferred to a larger mixer and 83.30 kg of microcrystalline cellulose are added, mixture proceeds during 20 minutes, the mixture is sieved through a US 30/40 mesh and then it is compressed into oblong shape (10×5) tablets of 350/400 mg, hardness 3-5 Kp, friability of less than 1.2% and disgregation time shorter than 20 sec.

### Example 14

### Tablet Composition No. 14 Elaboration

A premix is prepared by mixing in an small mixer 3 kg of sodium N-lauryl sarcosinate, 0.05 powder sodium monofluor fosfate, 0.1 kg of Triclosan, 11.30 kg of microcrystalline cellulose, 2 kg of sodium crosscarmellose, 1.5 kg of eucalyptus in microcrystalline cellulose, 2 kg of sodium saccharine and 0.05 kg of green pigment, during 15-20 minutes. The resulting premix is transferred to a larger mixer and 80 kg of microcrystalline cellulose are added, mixture proceeds during 20 minutes. The mixture is sieved through a US 60/100 mesh and then it is compressed into 13 mm diameter tablets of 720/750 mg, hardness 4-7 Kp, friability of less than 1% and disgregation time shorter than 15 sec.

### Example 15

### Tablet Composition No. 15 Elaboration

A premix is prepared by mixing in an small mixer 0.2 kg of hexetidine, 0.1 of zinc lactate, 8.68 kg of lactose, 3 kg of micronised sodium bicarbonate, 1 kg of sodium crosscarmellose, 3 kg of citrus extract in microcrystalline cellulose, 0.5 kg of micronised menthol and 0.02 kg of green pigment, during 15-20 minutes. The resulting premix is transferred to a larger mixer and 76 kg of microcrystalline cellulose, 2 kg of sodium lauryl sulfate, 2 kg of sodium saccharine, 3 kg of sodium bicarbonate, these are mixed for 20-30 minutes and the resulting composition is sieved through US 60 mesh.

Then it is compressed into 13 mm diameter tablets of 800 mg, hardness 4-7 Kp, friability of less than 1% and disgregation time shorter than 20 sec.

### Example 16

### Tablet Composition No. 16 Elaboration

A premix is prepared by means of a small mixer, wherein said premix comprises: 25-5 kg of microcrystalline cellulose, 2.5 kg of PVP-I, 4 kg of alantoine, 1 kg of fine powder salicylic acid. The resulting premix is poured into a pony mixer type vertical kneader and a liquid mixture comprising 5 kg of hydroalcoholic betula alba extract, 2.8 l of distilled water and 6 l of ethanol. This is kneaded during 30′ until a semi-wet homogeneous mass is obtained.

The resulting mass is granulated in a granulating machine with an outlet of US 16/18 mesh. The resulting granulate is conveniently distributed on stainless steel trays and is dried in hot air oven at 55/60° C. for 2/3 hours or until powder humidity is lower than 1.5%.

Once this new premix is obtained, its is crushed in a hammer type mill or the like until a fine powder with granules smaller than 180 microns is obtained.

The resulting powder is introduced into a double cone mixer with the addition of 52 kg of microcrystalline cellulose, 2 kg of sodium crosscarmellose and 8 kg of sodium cocoyl isethionate and this is mixed for 30 minutes. The resulting mixture is sieved through US 40 mesh and then it is compressed into tablets of 13 mm diameter, 720/750 mg, hardness 4-7 Kp, friability less than 1.2% and disgregation time of 20 sec or less.

### Example 17

### Tablet Composition No. 17 Elaboration

Quality control tests were performed for all of the raw materials—as in the previous formulations. This case particularly involves conditioning for the use of betamethasone dipropionate and clotrimazole and precautions as regards gentamicine. A premix is prepared by means of a small mixer, said premix comprises: 10.4 kg of microcrystalline cellulose, 3 kg of polyvinylpyrrolidone (PVP-K30); 0.1 kg of powder gentamicine, 1 kg of dry powder clotrimazole, 0.5 kg of dry powder betamethasone dipropionate, mixing amounting to 20/25 minutes. Premix is transferred to a larger mixer and 81 kg of microcrystalline cellulose and 4 kg of sodium cocoyl isethionate are mixed during 30 minutes and the resulting mixture is sieved through US mesh 30.

Subsequently the above is compressed into tablets of 13 mm diameter, 750-780 mg, hardness of 5-8 Kp, friability of less than 1% and disgregation time lower than 15 sec.

### Example 18

### Tablet Composition No. 18 Elaboration

A premix is prepared which is mixed in a small mixer and comprises: 12.3 kg of microcrystalline cellulose, 3 kg of starch (starch 1500), 0.2 kg of powder chlorhexidine diglutamate, 0.5 kg of salicylic acid, 6 kg of dry powder mandelic acid, 2 kg of aloe oil previously absorbed in microcrystalline cellulose, during 20/25 minutes. The resulting premix is transferred to a larger mixer adding 70 g of microcrystalline cellulose and 6 kg of sodium cocoyl isethionate, mixing proceeds during 20/25′. The resulting powder is sieved through US 30 mesh and then it is compressed into tablets of 13 mm diameter, 720/750 mg, hardness 4/7 Kp, friability of less than 1.2% and disgregation time shorter than 20 seconds.

### Example 19

### Tablet Composition No. 19 Elaboration

A wet granulation process is carried out, the following components being mixed: 15.9 kg of microcrystalline cellulose, a liquid solution of 4.6 l of ethanol and 0.1 kg of No. 4 Blue aluminum lake, 1 kg of violet oil (perfume), 2 kg of soybean oil and 11 kg of DEET. These are mixed and kneaded during 20′ in order to obtain an homogeneous mass, granulation is carried out through US 16 mesh and pouring into a fluidized bed during 1 hr at 55/60° C. and/or until drying to an humidity of 1.5% or less is obtained.

The resulting granulate is crushed in a hammer mill and using an outlet mesh such as to obtain a 120/150 microns granulation. The resulting powder is transferred to a mixer and blended with 60 kg of microcrystalline cellulose, 3 kg of sodium crosscarmellose and 7 kg of sodium lauryl sulfate during 20/30 minutes. Sieving is done through US 30/40 mesh and powder is compressed into tablets of 19 mm diameter, weight 1.5 g, hardness 5-8 Kp, friability lower than 1.5% and disgregation time lower than 20 sec.

### Example 20

### Tablet Composition No. 20 Elaboration

A wet granulation process is carried out by mixing the following components with 4.3 l of ethanol, 15 kg of Picaridine, 3 kg of P-menthanediol; 1 kg of eucalyptus-lemon and 0.1 kg of dye with 14.9 kg of microcrystalline cellulose. The mixture is kneaded during 20′ until homogenisation thereof and then it is passed through a US 16 mesh granulator. The resulting product is introduced into a fluidized bed dryer during 1 hr at 55/60° C. or until a powder humidity of 1.5% or less is obtained. The resulting powder is crushed to a particle size distribution of 90/120 microns and then it is transferred to a double cone mixer adding 60 kg of microcrystalline cellulose, 3 kg of sodium crosscarmellose and 3 kg of sodium lauryl sulfate while mixing for 30′. The resultant mixture is sieved through US 40 mesh and compressed into tablets of 19 mm diameter, 1.5/1.8 g, hardness 5-8 Kp, friability of less than 1.5% and disgregation time shorter than 20 sec.

### Example 21

### Tablet Composition No. 21 Elaboration

A wet granulation process is carried out by mixing in an adequate container, at room temperature: 3.5 l of ethanol with 10 kg of DEET, 2 kg of aloe vera oil, 2 kg of soybean oil. Said solution is poured over 15 kg of microcrystalline cellulose in a vertical kneader and mixing is performed until homogenisation during 20/25′. The resulting mass is sieved through US 16/18 mesh and placed on stainless steel trays which are introduced into a drying furnace during 1 hr at 60° C. The later is crushed in a hammer mill with an outlet mesh of US 100. Powder is transferred to a double cone mixer with the addition of 61 kg of microcrystalline cellulose, 3 kg of sodium crosscarmellose and 7 kg of sodium cocoyl isethionate, mixing is carried out during 30′. All of the resultant mixture is sieved through US 30 mesh and compressed into tablets of 13 mm diameter, hardness 4-7 Kp, weight 720/750 mg, friability of 1.2% or less and disgregation time lower than 15 sec.

### Example 22

### Tablet Composition No. 22 Elaboration

A premix is made by means of a small mixer with 13.5 kg of microcrystalline cellulose, 2 kg of liquid permetrine and 1.5 kg of liquid lindane previously absorbed in a portion of microcrystalline cellulose, mixing is performed for 15-20′ and if necessary the above is transferred to a larger mixer and 70 kg of microcrystalline cellulose, 4 kg of micronised citric acid, 3 kg of CMC and 6 kg of sodium cocoyl isethionate are added, mixing proceeds during 25/30 minutes and the resulting powder is sieved through US 30 mesh. Then compression is performed into tablets with 13 mm diameter, 720/750 mg, hardness 4-7 Kp, friability 1.2% or less and disintegration time shorter than 20 sec.

1. Solid tablet composition of quick dissolution in water for body and mouth cleansing, skin treatment and disinfection, characterised in that it comprises from 75% to 92% of at least one disintegrating agent, from 0.5% to 9% of at least one surfactant and from 0.5% to 4% of at least one disgregating agent.

2. The composition according to claim 1, characterised in that disintegrating agent is selected from the group comprised of microcrystalline cellulose, amylose, cellactose, powder cellulose, sugars, aerosiles and combinations thereof.

3. The composition according to claim 2, characterised in that sugars are selected from the group comprised by lactose, dextrose, glucose, sucrose and combinations thereof.

4. The composition according to claim 1, characterised in that the surfactant is selected from the group comprised of non ionic, anionic, cationic and amphoteric surfactants.

5. The composition according to claim 4, characterised in that the surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, cetrimide, sodium cocomonoglyceride sulfanate and combinations thereof.

6. The composition according to claim 1, characterised in that the disgregating agent is selected from the group comprised of crosscarmellose, carboximethylcellulose, carbopol, polyvinylpyrrolidone, starch, carrageenan and combinations thereof.

7. The composition according to claim 1, characterised in that it further comprises those products selected from the group comprised of vegetal oils, antibacterial agents, anti-inflammatory agents, plant extracts, insecticides, pH buffers, sweeteners, flavours, fluorides, vitamins, dyes, insect-repelling agents, perfumes, antiperspirant agents, antifungal agents, decongestive agents, abrasives, disinfectants, peptides, effervescing agents, tartar inhibitors, and combinations thereof.

8. The composition according to claim 7, characterised in that vegetal oils are selected from the group comprised of violet oil, eucalyptus oil, lemon oil, aloe vera oil, algae oils, rose oil, lavender oil, rosemary oil, almond oil, green tea oil, coconut oil, olive oil, calendula oil, jojoba oil, salvia oil, arnica oil, strawberry oil, complexes and oily extracts and combinations with the above.

9. The composition according to claim 7, characterised in that disinfectant agents are selected from the group comprised of triclosan, chlorhexidine, iodine polyvinylpyrrolidone, benzalkonium chloride, cetrimide (cetyltrimethylammonium bromide), sodium N-lauryl sarcosinate.

10. The composition according to claim 7, characterised in that the anti-inflammatory agent is selected from the group comprised of alantoine, salicylic acid, calamine, mandelic acid, licorice extract, Arnica Montana, camomile, Hammamelis Virginiana.

11. The composition according to claim 7, characterised in that sweetener is selected from the group comprised of sodium saccharine, aspartame, lactose, dextrin, cyclamates, xylitol and sucrose.

12. The composition according to claim 7, characterised in that the insect-repelling agent is selected from the group comprised of picaridine, P-menthanediol (PMD), DEET (N,N-diethyl-toluamide), soybean oil and citronella.

13. The composition according to claim 7, characterised in that the anti-fungal agent is selected from the group comprised of miconazole, clotrimazole, miconazole nitrate, isoconazole nitrate, betamethasone dipropionate, lauryldimethylbenzylammonium chloride, benzoic acid, boric acid, benzalkonium chloride.

14. The composition according to claim 7, characterised in that the antibacterial agent is selected from the group comprised of antibiotics and bacteriostatic agents.

15. The composition according to claim 7, characterised in that fluorides are selected from the group comprised of nicomethanol fluorhydrate, sodium monofluor fosfate, tin fluoride, sodium fluoride, potassium fluoride and amine fluor.

16. The composition according to claim 7, characterised in that vitamins are selected from the group comprised of panthenol, dexpanthenol, retinol, carotene, folic acid, DL-alfa-tocopherol, ascorbic acid, nicotinamide, biotin and colina.

17. The composition according to claim 7, characterised in that insecticides are selected from the group comprised of lindane, permethrin, ivermectin, pyrethrin, malathion, piperonil butoxide.

18. The composition according to claim 7, characterised in that antiperspirant agents are selected from the group comprised of aluminum hydrochloride, cyclometicone, aluminum-zirconium hydrochloride, salvia officinalis oil.

19. The composition according to claim 7, characterised in that abrasive agents are selected from group comprised of micronised sodium bicarbonate, calcium carbonate, sodium benzoate, sodium fosfate, alumina, silicates, zinc lactate.

20. The composition according to claim 7, characterised in that tartar formation inhibitors are selected from the comprised of hexetidine, Triclosan, chlorhexidine, sanguinarine, zinc citrate, sodium lauryl sulfate, fluorides.

21. Solid tablet composition of quick dissolution in water for body cleansing, characterised in that it comprises from 75% to 92% of at least one disintegrating agent, from 0.5% to 9% of at least one surfactant and from 0.5% to 4% of at least one disgregating agent.

22. The composition according to claim 21, characterised in that the disintegrating agent is selected from the group comprised of microcrystalline cellulose, amylose, cellactose, powder cellulose, sugars and combinations thereof.

23. The composition according to claim 22, characterised in that sugars are selected from the group comprised of lactose, dextrose, glucose, sucrose and combinations thereof.

24. The composition according to claim 21, characterised in that the surfactant is selected from the group comprised of non ionic, anionic, cationic and amphoteric surfactants.

25. The composition according to claim 24, characterised in that the surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, and combinations thereof.

26. The composition according to claim 21, characterised in that the disgregating agent is selected from the group comprised of crosscarmellose, carboximethylcellulose, carrageenan, carbopol, polyvinylpyrrolidone, starch and combinations thereof.

27. The composition according to claim 21, characterised in that it further comprises products selected from the group comprised by vegetal oils, antibacterial agents, anti-inflammatory agents, plant extracts, dyes, perfumes, antiperspirant agents, antifungal agents, decongestive agents, abrasives, disinfectants, and combinations thereof.

28. The composition according to claim 27, characterised in that vegetal oils are selected from the group comprised of violet oil, eucalyptus oil, lemon oil, aloe vera oil, algae oils, rose oil, lavender oil, flowers oil and combinations thereof.

29. The composition according to claim 27, characterised in that disinfectant agents are selected from the group comprised of triclosan, chlorhexidine, iodine polyvinylpyrrolidone, benzalkonium chloride and cetrimide (cetyltrimethylammonium bromide).

30. The composition according to claim 27, characterised in that the anti-inflammatory agent is selected from the group comprised of alantoine, salicylic acid, calamine, mandelic acid, licorice extract, Arnica Montana, camomile, Hammamelis Virginiana and combinations thereof.

31. The composition according to claim 27, characterised in that the antifungal agent is selected from the group comprised of miconazole, clotrimazole, miconazole nitrate, isoconazole nitrate, betamethasone dipropionate, lauryldimethylbenzylammonium chloride, benzoic acid, boric acid, and benzalkonium chloride.

32. The composition according to claim 27, characterised in that the antibacterial agent is selected from the group comprised of antibiotics and bacteriostatic agents.

33. The composition according to claim 27, characterised in that antiperspirant agents are selected from the group comprised of aluminum hydrochloride, cyclometicone, Salvia officinalis oil, and aluminum-zirconium hydrochloride.

34. Solid tablet composition of quick dissolution in water for mouth cleansing, characterised in that it comprises from 75% to 92% of at least one disintegrating agent, from 0.5% to 9% of at least one surfactant and from 0.5% to 4% of at least one disgregating agent.

35. The composition according to claim 34, characterised in that the disintegrating agent is selected from the group comprised of microcrystalline cellulose, amylose, cellactose, powder cellulose, sugars and combinations thereof.

36. The composition according to claim 35, characterised in that sugars are selected from the group comprised of lactose, dextrose, glucose, sucrose and combinations thereof.

37. The composition according to claim 34, characterised in that the surfactant is selected from the group comprised of non ionic, anionic, cationic and amphoteric surfactants.

38. The composition according to claim 37, characterised in that the surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, and combinations thereof.

39. The composition according to claim 34, characterised in that the disgregating agent is selected from the group comprised of crosscarmellose, carboximethylcellulose, polyvinylpyrrolidone, starch and combinations thereof.

40. The composition according to claim 34, characterised in that it further comprises products selected from the group comprised by vegetable oils, antibacterial agents, disinfectants, plant extracts, sweeteners, flavours, fluorides, vitamins, dyes, abrasive agents, effervescing agents, tartar inhibitors and combinations thereof.

41. The composition according to claim 40, characterised in that vegetable oils are selected from the group comprised of strawberry oil, eucalyptus oil and lemon oil.

42. The composition according to claim 40, characterised in that disinfectant agents are selected from the group comprised of triclosan, chlorhexidine, hexetidine, sanguinarine and essential oils.

43. The composition according to claim 40, characterised in that sweetener is selected from the group comprised of sodium saccharine, aspartame, lactose, dextrin, cyclamates, glucose, xylitol and sucrose.

44. The composition according to claim 40, characterised in that the antibacterial agent is selected from the group comprised of antibiotics and bacteriostatic agents.

45. The composition according to claim 40, characterised in that fluorides are selected from the group comprised of nicomethanol fluorhydrate, sodium monofluor fosfate, tin fluoride, sodium fluoride, potassium fluoride and amine fluor.

46. The composition according to claim 40, characterised in that vitamins are selected from the group comprised of provitamin B5, vitamin P, vitamin E and vitamin B5.

47. The composition according to claim 40, characterised in that abrasives are selected from the group comprised of micronised sodium bicarbonate, calcium carbonate, sodium benzoate, sodium fosfate, silicates and aluminum lactate.

48. The composition according to claim 40, characterised in that tartar formation inhibitors are selected from the comprised of hexetidine, zinc citrate, Triclosan, chlorhexidine, sanguinarine, essential oils, sodium lauryl sulfate, and fluorides.

49. Solid tablet composition of quick dissolution in water for skin treatment, characterised in that it comprises from 75% to 92% of at least one disintegrating agent, from 0.5% to 9% of at least one surfactant and from 0.5% to 4% of at least one disgregating agent.

50. The composition according to claim 49, characterised in that disintegrating agent is selected from the group comprised of microcrystalline cellulose, amylose, cellactose, powder cellulose, sugars, and combinations thereof.

51. The composition according to claim 50, characterised in that sugars are selected from the group comprised of lactose, dextrose, glucose, sucrose and combinations thereof.

52. The composition according to claim 50, characterised in that surfactant is selected from the group comprised from non ionic, anionic, cationic and amphoteric surfactants.

53. The composition according to claim 52, characterised in that the surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate and combinations thereof.

54. The composition according to claim 49, characterised in that disgregating agent is selected from the group comprised of crosscarmellose, carboximethylcellulose, carrageenan, carbopol, polyvinylpyrrolidone, starch and combinations thereof.

55. The composition according to claim 49, characterised in that it further comprises those products selected from the group comprised of vegetal oils, antibacterial agents, antiinflammatory agents, plant extracts, antifungal agents, decongestive agents, and combinations thereof.

56. The composition according to claim 55, characterised in that vegetal oils are selected from the group comprised of violet oil, eucalyptus oil, lemon oil, aloe vera oil, fucus oil, almond oil, coconut oil, olive oil, jojoba oil, green tea oil, rose oil, lavender oil, sandalwood oil, arnica oil, calendula oil, camomile oil, rosemary oil, salvia oil and combinations thereof.

57. The composition according to claim 55, characterised in that the anti-inflammatory agent is selected from the group comprised of alantoine, salicylic acid, calamine, mandelic acid, licorice extract, Arnica Montana, camomile, Hammamelis Virginiana.

58. The composition according to claim 55, characterised in that the antifungal agent is selected from the group comprised of miconazole, clotrimazole, miconazole nitrate, isoconazole nitrate, betamethasone dipropionate, lauryldimethylbenzylammonium chloride, benzoic acid, boric acid, and benzalkonium chloride.

59. The composition according to claim 55, characterised in that the antibacterial agent is selected from the group comprised of antibiotics and bacteriostatic agents.

60. Solid tablet composition of quick dissolution in water for insect repelling, characterised in that it comprises from 75% to 92% of at least one disintegrating agent, from 0.5% to 9% of at least one surfactant and from 0.5% to 4% of at least one disgregating agent.

61. The composition according to claim 60, characterised in that disintegrating agent is selected from the group comprised of microcrystalline cellulose, amylose, cellactose, powder cellulose, sugars, and combinations thereof.

62. The composition according to claim 61, characterised in that sugars are selected from the group comprised of lactose, dextrose, glucose, sucrose and combinations thereof.

63. The composition according to claim 60, characterised in that surfactant is selected from the group comprised of non-ionic, anionic, cationic and amphoteric surfactants.

64. The composition according to claim 63, characterised in that surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, and combinations thereof.

65. The composition according to claim 60, characterised in that the disgregating agent is selected from the group comprised of crosscarmellose, carboximethylcellulose, carrageenan, carbopol, polyvinylpyrrolidone, starch and combinations thereof.

66. The composition according to claim 60, characterised in that it further comprises those products selected from vegetal oils, plant extracts, insect repelling agents, perfumes, dyes and combinations thereof.

67. The composition according to claim 66, characterised in that vegetal oils are selected from the group comprised of violet oil, eucalyptus oil, lemon oil, aloe vera oil, algae oils, rose oil, lavender oil, soybean oil, salvia oil, calendula oil, jojoba oil and almond oil.

68. The composition according to claim 66, characterised in that insect-repelling agent is selected from the group comprised of picaridine, p-methanediol (PMD), DEET (N,N-diethyl-m-toluamide), soybean oil and citronella.

69. Solid tablet composition of quick dissolution in water for disinfection and pediculosis treatment, characterised in that it comprises from 75% to 92% of at least one disintegrating agent, from 0.5% to 9% of at least one surfactant and from 0.5% to 4% of at least one disgregating agent.

70. The composition according to claim 69, characterised in that disintegrating agent is selected from the group comprised of microcrystalline cellulose, amylose, cellactose, powder cellulose, sugars and combinations thereof.

71. The composition according to claim 70, characterised in that sugars are selected from the group comprised of lactose, dextrose, glucose, sucrose and combinations thereof.

72. The composition according to claim 69, characterised in that the surfactant is selected from the group comprised of non ionic, anionic, cationic and amphoteric surfactants.

73. The composition according to claim 72, characterised in that the surfactant is selected from the group comprised of sodium lauryl sulfate, sodium cocoyl isethionate, sodium cocomethyl taurate, sodium lauryl glutamate, ammonium lauryl sulfate, sodium cetearyl sulfate, sodium N-lauryl sarcosinate, cetrimide, sodium cocomonoglyceride sulfanate and combinations thereof.

74. The composition according to claim 69, characterised in that the disgregating agent is selected from the group comprised of crosscarmellose, carboximethylcellulose, polyvinylpyrrolidone, starch and combinations thereof.

75. The composition according to claim 69, characterised in that it further comprises products selected from the group comprised of vegetal oils, plant extracts, insecticides, pH buffers, dyes, perfumes, and combinations thereof.

76. The composition according to claim 75, characterised in that vegetable oils are selected from the group comprised of tea-branch oil, eucalyptus oil, citrus medica oil, aloe vera oil, algae oils, rosemary oil, calendula oil, soybean oil, almond oil, lavender oil and combinations thereof.

77. The composition according to claim 75, characterised in that insecticides are selected form the group comprised of lindane, permethrin, ivermectin, malathion, pyrethrin and piperonil butoxide.
